(19) United States
(12) Patent Application Publication
(10) Pub. No.: US 2003/0055368 A1
(43) Pub. Date: Mar. 20, 2003

(54) Heel treatment device and method

(76) Inventor: Richard Jacoby, Paradise Valley, AZ (US)

Correspondence Address: WEISS & MOY PC, 4204 NORTH BROWN AVENUE, SCOTTSDALE, AZ, 85251, US

(21) Appl. No.: 09/954,818

(22) Filed: Sep. 19, 2001

(51) Int. Cl. A61F 5/00

(52) U.S. Cl. 602/27

(57) ABSTRACT

A heel treatment device and method, which consists primarily of a shaped area dimensioned to conform to the shape of a human heel and a strap portion dimensioned to be positioned over the top portion of the foot. A topical application for treatment of a condition of a human heel is positioned on the heel or on the interior of the shaped portion, and the device is placed on the foot of the wearer. Preferably, an applicator is also positioned in the interior portion of the shaped area. It is further preferred that the strap portion consist of two straps adapted to be releasably coupled together by the positioning of hook and loop material on end portions thereof.

### BACKGROUND OF THE INVENTION

### Field of Invention

This invention relates generally to devices and methods for treating heels and, more particularly, to a device and method for providing heel treating cream to an affected area in a manner that keeps the cream in contact with the heel, that promotes comfort, and that is relatively clean and neat.

### BACKGROUND OF THE INVENTION

There are a number of conditions that affect the human heel. These include dry and cracked skin, callouses, xerosis and fissuring. To address these, it is generally preferred to apply a topical application of some kind to the affected area. In order to keep the application in contact with the heel, patients sometimes will wrap the area in Saran Wrap. However, such a wrapping can be cumbersome, inconvenient, and not particularly effective at keeping the cream in place in a clean manner.

A need therefore existed for a device and method for facilitating the application of a topical solution to an affected heel in a manner that is relatively clean, maintains the cream in contact so that it can be effective, and is comfortable for the user. The present invention satisfies these needs and provides other, related, advantages.

### SUMMARY OF THE INVENTION

It is an object of the present invention to provide a heel treatment device and method that facilitates the application of a topical solution to an affected heel in a manner that is relatively clean.

It is a further object of the present invention to provide a heel treatment device and method that is effective at maintaining treating cream in contact with an affected heel.

It is a still further object of the present invention to provide a heel treatment device and method that is comfortable with the user.

### BRIEF DESCRIPTION OF THE PREFERRED EMBODIMENTS

In accordance with one embodiment of the present invention, a heel treatment device is disclosed. The heel treatment device comprises, in combination: a shaped area dimensioned to be positioned over a human heel of a human foot and to maintain a topical application for treatment of the human heel in contact with the human heel; a strap portion coupled to the shaped area and dimensioned to be positioned over a top portion of the human foot while permitting a toe portion of the human foot to extend through an opening formed by a front edge portion of the shaped area and a front edge portion of the strap portion so that the toe portion of the human foot is exposed.

In accordance with another embodiment of the present invention, a method for treating a heel is disclosed. The method comprises the steps of: providing a heel treatment device comprising, in combination: a shaped area dimensioned to be positioned over a human heel of a human foot and to maintain a topical application for treatment of the human heel in contact with the human heel; a strap portion coupled to the shaped area and dimensioned to be positioned over a top portion of the human foot while permitting a toe portion of the human foot to extend through an opening formed by a front edge portion of the shaped area and a front edge portion of the strap portion so that the toe portion of the human foot is exposed; applying the topical application to one of the shaped area and the human heel; positioning the shaped area over the human heel; and positioning the strap portion over the top portion of the human foot.

The foregoing and other objects, features, and advantages of the invention will be apparent from the following, more particular, description of the preferred embodiments of the invention, as illustrated in the accompanying drawings.

### BRIEF DESCRIPTION OF THE DRAWINGS

FIG. 1 is a perspective view of an embodiment of the heel treatment device of the present invention, in position on a human foot.

FIG. 2 is a perspective view of an embodiment of the heel treatment device of the present invention, shown separate from a human foot.

FIG. 3 is a perspective view of the heel treatment device of FIG. 2, illustrating how it may be coupled to a human foot.

FIG. 4 is a perspective view of an applicator which, in one embodiment of the present invention, is positioned within the heel treatment device.

### DETAILED DESCRIPTION OF THE PREFERRED EMBODIMENTS

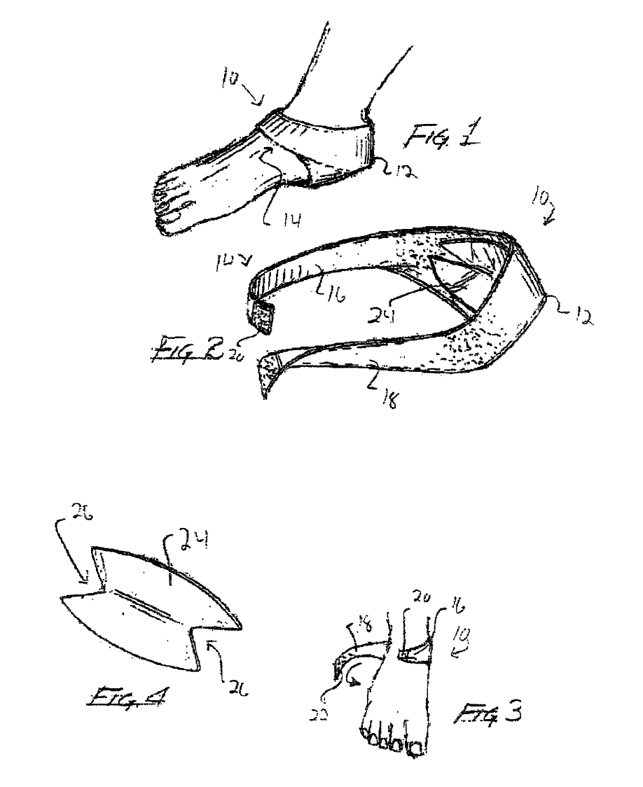

Referring first to FIGS. 1-3, the heel treatment device 10 of the present invention is shown. The heel treatment device 10 has a shaped area 12, which is dimensioned to conform to the shape of a human heel so that when positioned over a human heel (as shown in FIGS. 1 and 3), an interior portion of the shaped area 12 should fit the heel in a relatively snug manner. Referring now to FIG. 1, the heel treatment device 10 further features a strap portion 14, which is adapted to be positioned over a top portion of a human foot. The length of the strap portion 14 should be such so that, when positioned over the top portion of a human foot, the shaped area fits in a relatively snug and comfortable manner over the heel.

It is preferred that the shaped area 12 and strap portion 14 be formed from a single piece of material, although it would certainly be possible to form them separately and coupled them together in some appropriate manner—by sewing, sealing, or some other means. It is further preferred that the shaped area 12 and strap portion 14 be machine washable so that the heel treatment device 10 may be re-used, but non-washable, disposable material would be suitable for single treatment use.

The strap portion 14 may be continuous, so that the heel treatment device 10 would have to be slipped over the human foot in a sock-like manner. In such instance, it would likely be necessary to form the strap portion 14—and perhaps also the shaped area 12—from a material having elastic qualities.

Referring now to FIGS. 2-3, it would be preferable for the strap portion 14 to comprise a first strap 16 and a second strap 18. In such a configuration, it is necessary to provide means proximate an end portion of the first strap 16 and second strap 18 to permit coupling therebetween. The preferred means is hook and loop material, with a first coupling area 20 comprising either hook or loop material located on an outside, end portion of the first strap 16, and a second coupling area 22 on an inside, end portion of the second strap 18, comprising hook or loop material opposite that positioned on the first strap 16 To couple the first strap 16 to the second strap 18—and thereby to securely position the heel treatment device 10 on the foot—the first strap 16 would be positioned over the top portion of the foot with the first coupling area 20 exposed, aid the second strap 18 would then be positioned so that the second coupling area 22 contacts the first coupling area 20.

While the use of hook and loop material would be preferred—particularly in a re-useable version of the heel treatment device 10—other connecting means could be utilized. For example, and particularly in a disposable variant of the heel treatment device 10, tape or other adhesive could be utilized.

Referring now to FIGS. 2 and 4, it is preferred that the heel treatment device 10 further include an applicator 24, which applicator 24 would be positioned in the interior of the shaped area 12. The underside of the applicator 24 may be affixed to the interior of the shaped area 12 by gluing or other means. As shown in FIG. 4, the applicator 24 preferably has indented regions 26 on both sides thereof, so that when the shaped area 12 is positioned around the human heels the applicator 24 may envelop the heel without the applicator 24 forming bunching regions along the sides thereof. It is preferred that the applicator 24 be formed from cotton or like material, but a wide variety of other materials used therapeutically—such as gauze and the like—could also be employed.

The applicator 24 may be permanently affixed to the interior portion of the shaped area 12. This configuration may be preferred where the device 10 is disposable. However, where the device 10 is configured to be re-useable, it would be preferred to make the applicator 24 readily removable from the shaped area 12—so that the applicator 24 may be disposed of following use, while the remainder of the device 10 is washed and then fitted with a replacement applicator 24 for future use.

It should be noted that the applicator 24 could be impregnated with a topical application and provided in this manner to the user, so as to eliminate any need on the part of the user to undertake the separate step of applying the topical application.

To use the heel treatment device 10, the user would apply a topical application either to the surface of the heel or to the interior of the shaped area 12. It the heel treatment device 10 includes an applicator 24, the topical application could be applied directly to the exposed surface of the applicator 24—by the user or by the manufacturer during assembly of the heel treatment device 10.

The heel treatment device 10 should then be positioned on the foot of the wearer, with the interior portion of the shaped area 12 being placed over the heel and the strap portion 14 being placed over the top portion of the foot. Where the strap portion 14 comprises a first strap 16 and a second strap 18, these should be coupled to one another in a relatively secure and comfortable manner as discussed above.

As shown in FIGS. 1 and 3, a front edge portion of the shaped area 12 and a front edge portion of the strap portion 14 together form an opening. This permits the toe portion of the human foot to extend therethrough and be exposed. This configuration is advantageous. By covering only a relatively small amount of the foot, the heel treatment device 10 should not unduly interfere with the user's wearing of socks and/or shoes together with the heel treatment device 10. Additionally, the heel treatment device 10 is made more comfortable, and should not unduly raise the temperature of the foot or otherwise interfere with sleep.

While the invention has been particularly shown and described with reference to preferred embodiments thereof, it will be understood by those skilled in the art that the foregoing and other changes in form and details may be lade therein without departing from the spirit and scope of the invention.

I claim:

1. A heel treatment device comprising, in combination:
a shaped area dimensioned to be positioned over a human heel of a human foot and to maintain a topical application for treatment of said human heel in contact with said human heel;
a strap portion coupled to said shaped area and dimensioned to be positioned over a top portion of said human foot while permitting a toe portion of said human foot to extend through an opening formed by a front edge portion of said shaped area and a front edge portion of said strap portion so that said toe portion of said human foot is exposed.

2. The heel treatment device of claim 1 further comprising an applicator positioned in said interior portion of said shaped area.

3. The heel treatment device of claim 2 wherein said applicator has indented regions formed on sides thereof.

4. The heel treatment device of claim 1 wherein said strap portion comprises two straps dimensioned to be pulled over said top portion of said human foot and coupled together.

5. The heel treatment device of claim 4 wherein one said strap comprises one of hook and loop material located proximate an end portion thereof and the other said strap comprises the other of said hook and said loop material proximate an end portion thereof so that said straps may be removably coupled together by bringing said one of hook and said loop material in contact with said other of said hook and said loop material.

6. The heel treatment device of claim 2 wherein said strap portion comprises two straps dimensioned to be pulled over said top portion of said human foot and coupled together.

7. The heel treatment device of claim 6 wherein one said strap comprises one of hook and loop material located proximate an end portion thereof and the other said strap comprises the other of said hook and said loop material proximate an end portion thereof so that said straps may be removably coupled together by bringing said one of hook and said loop material in contact with said other of said hook and said loop material.

8. A method for treating a heel comprising the steps of:
providing a heel treatment device comprising, in cormbination.
a shaped area dimensioned to be positioned over a human heel of a human foot and to maintain a topical application for treatment of said human heel in contact with said human heel;
a strap portion coupled to said shaped area and dimensioned to be positioned over a top portion of said human foot while permitting a toe portion of said human foot to extend through an opening formed by a front edge portion of said shaped area and a front edge portion of said strap portion so that said toe portion of said human foot is exposed;
applying said topical application to one of said shaped area and said human heel;
positioning said shaped area over said human heel; and
positioning said strap portion over said top portion of said human foot.

9. The method of claim 8 further comprising the step of positioning an applicator in said interior portion of said shaped area.

10. The method of claim 9 wherein said applicator has indented regions formed on sides thereof.

11. The method of claim 8 wherein said strap portion comprises two straps dimensioned to be pulled over said top portion of said human foot and coupled together.

12. The method of claim 11 wherein one said strap comprises one of hook and loop material located proximate an end portion thereof and the other said strap comprises the other of said hook and said loop material proximate an end portion thereof so that said straps may be removably coupled together by bringing said one of hook and said loop material in contact with said other of said hook and said loop material.

13. The method of claim 9 wherein said strap portion comprises two straps dimensioned to be pulled over said top portion of said human foot and coupled together.

14. The method of claim 13 wherein one said strap comprises one of hook and loop material located proximate an end portion thereof and the other said strap comprises the other of said hook and said loop material proximate an end portion thereof so that said straps may be removably coupled together by bringing said one of hook and said loop material in contact with said other of said hook and said loop material.
